(19) United States
(12) Patent Application Publication
(10) Pub. No.: US 2011/0054201 A1
(43) Pub. Date: Mar. 3, 2011

(54) Process for Producing Fatty Acid Esters and Fuels Comprising Fatty Acid Esters

(76) Inventors: Josip Kuftinec, Hrvatski Leskovac (HR); Nikola Blazevic, Zagreb (HR); Marijan Hohnjec, Zagreb (HR); Kata Blazevic, Zagreb (HR)

(21) Appl. No.: 12/377,982

(22) PCT Filed: Aug. 24, 2007
(86) PCT No.: PCT/HR2007/000025

§ 371 Date: Oct. 22, 2010

(30) Foreign Application Priority Data
Aug. 30, 2006 (HR) P20060287A

(51) Int. Cl. C07C 67/08

(52) U.S. Cl. 554/170

(57) ABSTRACT

A process for producing fatty acid esters with a high yield from an unrefined natural oil or fat, such as waste oil discarded by restaurant, food industries or common homes. The process comprises reacting an oil or fat with an alcohol in the presence of a titanate catalyst of general formula: Ti(OR)₄ in which R means: methyl, ethyl, isopropyl, n-buthyl, 2-ethylhexyl or octoleneglycole rest.

### CROSS-REFERENCE TO RELATED APPLICATIONS

This application represents a National Stage application of PCT/HR2007/000025 entitled “Process for Producing Fatty Acid Esters and Fuels Comprising Fatty Acid Esters” filed Aug. 24, 2007, pending.

### BACKGROUND OF THE INVENTION

1. Field of the Invention

The present invention relates to a process for producing a fatty acid ester and glycerol by reacting an oil or fat with an alcohol, and to a fuel comprising a fatty acid ester obtained by the above process.

2. Description of Related Art

The present invention relates to the production of methyl acid esters starting from unrefined natural oils or fats as well as waste oil discarded by restaurant, food industries or common homes.

It is well known that methyl esters of fatty acids become in the last decade very interesting. They are used mostly as biodiesel—the substitute for fossil fuel and as starting material for production of other derivatives of fatty acids such as alcohols and amides.

The process for industrial manufacture of the above mentioned esters is very simple and consists of the reaction of triglycerides (the main constituents of fats and oils) with methanol, in the presence of catalysts. In this reaction glycerin is obtained as by product. Glycerin is also useful as starting material for the preparation of other chemicals, and purified is a valuable component for pharmaceutical products. The production processes were developed during the last decade in order to improve the yield and economical efficiency as well as to make possible use of different starting materials. During the time the first used raw material—refined rapeseed oil has become too expensive and the raw material base was therefore extended.

In the classical manufacturing procedure, basic catalysts (NaOH, KOH, NaOCH₃) are applied for transesterification of triglycerides. From a chemical point of view these catalysts are very efficient, but they have several disadvantages [G. Vicent et al, Ind. Crops and Prod. 8 (1998), 29-35]:
Free fatty acids, which are always present in some extent in raw material, are consuming catalyst for neutralization, giving soaps. This by product means decreasing of yield by loosing of the part of raw material.
On the other hand, soaps are causing problems in downstream processing, especially glycerin purification. The most practical equipment for distillation of glycerin (thin-layer evaporators) is not applicable because of the presence of soaps and inorganic salts formed by neutralization of basic catalyst at the end of transesterification reaction. Difficulties in glycerin purification mean increasing the price of biodiesel. There is a possibility to remove inorganic salts by ion-exchange process, but again that means increasing of manufacturing costs and decreasing the efficiency of methanol recycling and the yield of glycerin.

A new process was then developed in order to avoid loosing of free fatty acid and to use raw materials with higher acids content. In World Patent Publication WO 01/12581, free fatty acids are in a first stage of process esterified by an acidic catalyst and, in a second stage, the acidic catalyst is removed by neutralization with a base, and afterwards with excess of base transesterification reaction is carried out. In this method an additional problem is removing of salt formed in neutralization of the acidic catalyst.

According to U.S. Pat. No. 6,399,800, esterification of the free fatty acids is solved in the following manner:

Saponification of total amount of material is carried out

Water is removed

Esterification of the dry saponification rest is carried out by adding corresponding alcohol and inorganic acid

Several methods for removal of free fatty acids before transesterification reaction are also known. These include separation of free fatty acids from raw oil by caustic washing, steam stripping and liquid extraction. The big disadvantage of caustic washing is loss of oil during the processing, which can amount twice to the amount of free fatty acid present in the starting raw oil.

There was always the interest to discover a catalyst and process which can accept a very wide range of raw materials with a rather high free fatty acids content and to carry out esterification and transesterification simultaneously.

In 1998 M. Diasakou at al. published an article [Fuel 77, 1297] which describes kinetics of transesterification of soybean oil at high temperatures (200-260° C.) without the presence of a catalyst. The reaction process is slow, lasts for 8-10 hours and the yield was around 80% of methyl esters.

In U.S. Patent Application Publication 2001/0042340, solid catalysts are used at high temperatures up to 260° C. It is a known process in which the transesterification is carried out under the following condition: pressure 9 to 10 MPa, temperature 230-260° C., and an oil of low purity containing free fatty acid can be used as a raw material [Ullmann Encyclopedia of Industrial Chemistry, Fifth Edition, Vol. A10 (1997) p. 281].

In the production of methyl esters for the preparation of polyester (polyoxyethylene glycol), the titanates, especially alcoxy titanates, are used as catalysts, deposited on montmorillonite clay used as a carrier (see U.S. Pat. No. 4,032,550).

### SUMMARY OF THE INVENTION

The present invention is, generally directed to a process for producing fatty acid esters with a high yield from an unrefined natural oil or fat, such as waste oil discarded by restaurant, food industries or common homes. The main aspect of the process concerns reacting an oil or fat with an alcohol, preferably under subcritical conditions in a reactor without stirring, in the presence of a titanate catalyst of general formula: Ti(OR)₄ in which R means: methyl, ethyl, isopropyl, n-buthyl, 2-ethylhexyl or octoleneglycole. The invention details the manner in which this process is carried out, as well as the pressure vessel arrangement in which the process is performed in accordance with a preferred embodiment of the invention.

### DESCRIPTION OF THE DRAWING

FIG. 1 is a schematic cross-sectional view of a pressure vessel employed in producing a fatty acid ester in accordance with the invention.

### DESCRIPTION OF THE INVENTION

Methyl fatty acid esters are prepared in accordance with the inventive process from fats and oils which also contain free fatty acids. In particular, the present invention relates to the preparation of methyl esters from mixtures of triglycerides and free fatty acids. Raw materials include unrefined oils like palm, soybean, coconut, rapeseed and waste, i.e., discarded frying oil as well. The process of esterification and transesterification according to the inventive process is carried out using methanol and titanate catalyst of general formula: Ti(OR)₄ in which R means: methyl, ethyl, isopropyl, n-buthyl, 2-ethylhexyl and octyleneglycole.

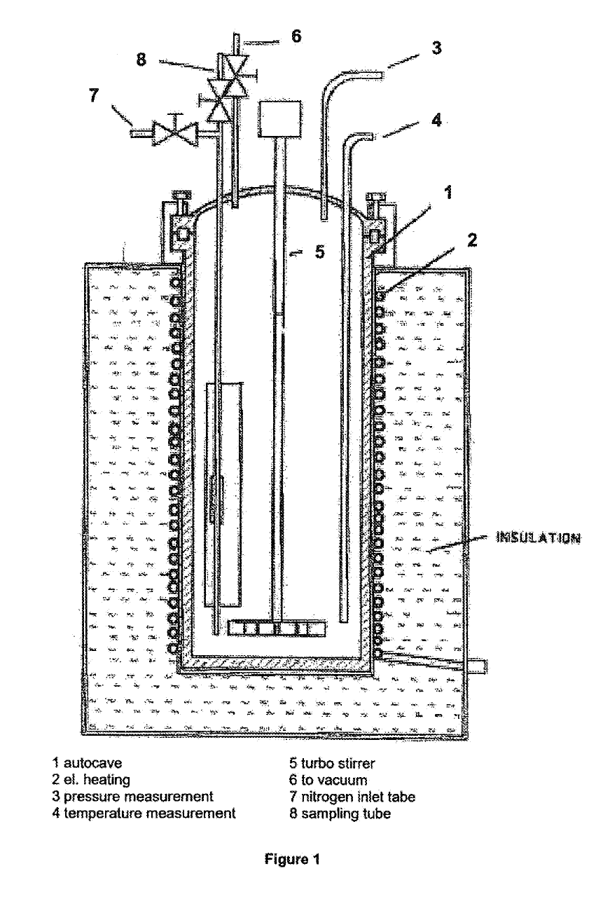

The reaction is carried out in a pressure vessel presented on the enclosed drawing (FIG. 1) under pressure of 28-60 bars and temperature 200-240° C. In the inventive process, all disadvantages present in previous processes using titanate catalyst are eliminated. The reaction is carried out preferably in one or two stages. By the efficient intermediate removing of water and glycerin, the catalyst action is not disturbed by the presence of those by-products and we realized the following targets:
The total reaction time is reduced to 60 minutes
Due to the gradual removing of glycerin and the adding fresh catalyst, the reaction is completed
Any pretreatment of raw material is not required
There is no limitation in using of raw materials, (oils with even 25% of free fatty acids are suitable for processing)

In the first stage oil is heated with 300-600 ppm of catalyst (preferably 500 ppm) and methanol is added (15-50 mol to 1 mol of oil).

Heating is carried out for 25-35 minutes (preferably 30 minutes) at 200-240° C. (preferably 230° C.). After cooling a reactions mixture to 50-60° C. on a rotary evaporator, 80-95% of a theoretical amount of glycerin is separated. In the second reaction stage, carried out under the same condition as first one, an additional 5-20% of glycerin is removed.

It is also possible to complete the reaction in two and even in one step, but with increased amount of methanol and catalyst.

It should be noted that in the present process free fatty acids do not form any soaps because they are converted 97% into methyl esters, Glycerin obtained is very pure and it is easy to get pharmaceutical quality by simple filtration (to remove TiO₂) and distillation under reduced pressure.

The inventive process is more closely illustrated, yet in no way limited by the following:

### EXAMPLES

### Example 1

This example illustrates esterification and transesterification reaction using soybean oil enriched with 7% of oleic acid:

Acid value 14.86

Water content 0.07%

Methanol used for reaction: anhydrous, water content less than 0.05%.

Catalyst: Mixture of Tetra alkyl titanates TYZOR TPT-20B with the following composition:

80% tetra isopropyl titanate

20% tetra n-butyl titanate

Gas chromatography analyses of products were performed on a Perkin Elmer 8700, gas chromatograph equipped with a modified on-column injector, flame-ionization detector and Rtx-1 (RESTEK) capillary column [15 m, 0.32 mm ID] coated with 0.10 μm film of 100% dimethyl polysiloxane.

### Procedure:

1. Stage: without Catalyst (Repeated Experiment According to the Article M. Diasakou et al.)

Into the reactor vol. 600 ml (presented in the attached drawing) about 220 g (0.25 mol) of soybean oil enriched with 7% oleic acid is added. After a few minutes 180 g (5.63 mol) of dry methanol is added and reactor is closed. The reaction mixture was heated 45 minutes under stirring (stirring speed 970-1020 rpm) until the reaction temperature of 230° C. is reached. Heating and stirring is continued for 30 minutes keeping the same temperature. The pressure in the reactor at the beginning of reaction was 45.5 bars and at the end decreases to 40 bars. After 30 minutes heating was stopped and reactor is cooled down to 50-60° C. Reaction mixture is transferred into 1 L round bottom flask and methanol is evaporated on rotary evaporator (bath temperature 85° C., 40 mbars pressure) within 40 minutes. After standing in separatory funnel for 30-40 minutes, glycerin (lower layer) is separated representing about 75% of theoretical amount of glycerin.

### Example 2

### 1. Stage

### With Catalyst

Into the reactor vol. 600 ml 220 g (0.25 mol) of rapeseed oil enriched with 7% oleic acid and 0.24 g (1000 ppm) catalyst TIZOR TPT-20B are added and the content mixed. After a few minutes 180 g (5.63 mol) of dry methanol is added, reactor is closed and heated for 45 minutes with stirring until reaction mixture achieved temperature of 230° C. Heating is continued for 30 minutes under the same temperature. The pressure in the reactor at the beginning of reaction is 45.5 bars and at the end decreases to 40 bars. After 30 minutes heating is stopped and reactor is cooled to 50-60° C., Reaction mixture is transferred into 1 L round bottom flask and methanol is removed by evaporation on rotary evaporator (bath temperature 85° C., 40 mbars pressure) within 40 minutes. After standing in separatory funnel for 30-40 minutes, glycerin (lower layer) is separated giving about 90% of theoretical amount of glycerin. Results are shown in Table 1.

### Example 3

In the identical way we performed esterification and transesterification with raw palm oil containing 25% of free fatty acids. In this experiment we changed methanol-oil molar ratio to 50:1. Results obtained are given in Table 1. It is noticeable that esterification and transesterification can be performed in one stage with yield over 90%.

### Example 4

The Example 2 is repeated, but with no stirring. Results obtained are identical to those in Example 2, which is surprising because the most of patents pointed out good stirring of reactants in reaction chamber. In our test the difference was not noticeable.

### Example 5

### 1. Stage

### With Catalyst

Into the reactor vol. 600 ml, 220 g (0.25 mol) of rapeseed oil enriched with 7% oleic acid, 0.24 g (500 ppm) catalyst TIZOR TPT-20B were added and the content mixed. After a few minutes 180 g (5.63 mol) of dry methanol is added and reactor is closed. The reaction mixture was heated for 45 minutes without stirring until temperature of 230° C. is achieved. Heating is continued for 30 minutes under the same temperature. The pressure in the reactor at the beginning of reaction is 45.5 bars and at the end decreases to 40 bars. After 30 minutes heating is stopped and reactor is cooled to 50-60° C. Reaction mixture is transferred into 1 L round bottom flask and methanol is removed by evaporation on rotary evaporator (bath temperature 85° C., 40 mbars pressure) within 40 minutes. After standing in separatory funnel for 30-40 minutes glycerin (lower layer) is separated giving about 90% of theoretical amount of glycerin.

In this test the quantity of catalyst was lowered due to lesser percentage of free fatty acids than in Example 3.

Results are shown in Table 1.

### 2 Stage

The upper layer (218 g) is transferred again into pressure reactor and 0.15 g of catalyst and 180 g of methanol is added. Reactor is closed and transesterification is carried out during 30 minutes at 230° C. The pressure at the beginning of reaction is about 48 bars and at the end decreases to 45 bars. After 30 minutes reaction product is treated in the same manner as in the 1. Stage. Byproduct is about 10% theoretical amount of glycerin. Results are shown in Table 1.

### Example 6

In this test molar ratio oil-methanol was 1:37. The goal was to finish reaction in first stage if possible. The oil used was the same as in Example 5. Obtained results show 62% improvement in yield, which means that higher quantity of methanol results in better yield. Negative side effects are decrease in effective, volume of reactor, and higher energy consumption in methanol regeneration.

### 1. Stage

Into the reactor vol. 600 ml 150 g (0.17 mol) of raw palm oil and 0.08 g (500 ppm) catalyst TIZOR TPT-20B are added and the content mixed. After a few minutes 200 g (6.26 mol) of dry methanol is added and reactor is closed. Heating is carried out within 30 minutes without stirring until reaction mixture has reached temperature of 230° C. Heating is continued for 30 minutes under the same temperature. Results are shown in Table 1.

### Esterification of Free Fatty Acid (FFA)

### Example 7

The esterification of FFA is possible under the same conditions as it is shown by batch D-130. Since esterification is very efficient, AV is reduced after first stage from 151.5 to 6.83 which means 95.5%. The amount of catalyst is 1000 ppm and oil (FFA)-methanol, ratio is 1:13 calculated as oleic acid. See Table 1.

### Flash Evaporation of Methanol

### Example 8

In the batches D-139 to D-141 direct evaporation of methanol is applied without opening the reactor.

Raw materials for second stage are also added without opening the reactor. The problem is removing glycerine from the reactor, since glycerin has a negative influence on completion of the second stage of reaction. In case of using a reactor with a bottom discharge, it will be possible to complete glycerine separation (lower layer) and consequently complete the reaction. Analytical results for 1. Stage: MG 4.99%; DO 0.24%; TG 0.57%, and for the 2. Stage; MG 1.51%; DG 0.11%; TG 0.01%. See Table 2.

TABLE 1
Results of esterification and transesterification of different oils

| | | | | Oil | | | | | | | | | |
|---|---|---|---|---|---|---|---|---|---|---|---|---|---|
| Example | | | Raw | amount | | MeOH | | Catalyst | | MG | DG | TG | |
| Nr. | Charge | Step | material | (g) | AV | (g) | Ratio | (ppm) | Mixing | (%) | (%) | (%) | AV |
| Example 1 | D-086 | 1 | Rafined | 220.23 | 14.86 | 175.8 | 22.05 | Cat. Is | yes | 11.56 | 3.98 | 0.7 | 1.85 |
| | D-087 | 1 | Soy oil (*) | 223.42 | 14.86 | 179.53 | 22.20 | Not used | yes | 13.87 | 9.3 | 3.47 | 0.52 |
| | D-088 | 1 | | 220.53 | 14.86 | 174.9 | 21.91 | | yes | 12.28 | 5.24 | 1.81 | 2.81 |
| Example 2 | D-90 | 1 | Rafined | 250.12 | 14.52 | 151.8 | 16.77 | 1000 | yes | 4.24 | 0.44 | 0.07 | 0.99 |
| | D-91 | 1 | rape oil (**) | 252.92 | 14.52 | 151.3 | 16.53 | 1000 | yes | 4.54 | 0.42 | 0.04 | 0.88 |
| | D-92 | 1 | | 250.8 | 14.52 | 150.8 | 16.61 | 1000 | yes | 4.07 | 0.51 | 0.28 | 1.01 |
| Example 3 | D-104 | 1 | Raw | 102.5 | 48.45 | 182.6 | 49.21 | 1000 | yes | 2.13 | 0.37 | 0.79 | 2.02 |
| | D-105 | 1 | palm oil (***) | 100.39 | 48.45 | 184.3 | 50.72 | 1000 | yes | 1.82 | 0.35 | 0.3 | 1.43 |
| | D-110 | 1 | | 106.19 | 48.45 | 185.2 | 48.18 | 1000 | yes | 2.78 | 0.95 | 0.26 | 1.94 |
| Example 4 | D-120 | 1 | Rafined | 250.06 | 14.52 | 150.1 | 16.58 | 1000 | no | 4.48 | 0.46 | 0.16 | 0.93 |
| | D-121 | 1 | rape oil (**) | 251.92 | 14.52 | 152.1 | 16.68 | 1000 | no | 4.83 | 0.55 | 0.47 | 0.99 |
| | D-122 | 1 | | 252.25 | 14.52 | 151.9 | 16.64 | 1000 | no | 4.42 | 0.79 | 0.71 | 0.88 |
| Example 5 | D-125 | 1 | Raw | 223.88 | 15.27 | 175.21 | 21.62 | 500 | no | 3.68 | 0.43 | 0.7 | 0.89 |
| | | 2 | palm oil # | 218.32 | 0.89 | 168.52 | 21.32 | 500 | no | 0.57 | 0 | 0 | 0.41 |
| | D-126 | 1 | | 220.18 | 15.27 | 175.23 | 21.99 | 500 | no | 3.52 | 0.18 | 0.03 | 0.82 |
| | | 2 | | 216.28 | 0.82 | 169.25 | 21.62 | 500 | no | 0.64 | 0 | 0 | 0.25 |
| | D-127 | 1 | | 225.12 | 15.27 | 175.23 | 21.50 | 500 | no | 3.95 | 0.07 | 0 | 0.82 |
| | | 2 | | 225.43 | 0.82 | 176.26 | 21.60 | 500 | no | 0.8 | 0.07 | 0 | 0.24 |
| | D-128 | 1 | | 222.18 | 15.27 | 178.23 | 22.16 | 500 | no | 3.1 | 0.17 | 0.05 | 0.82 |
| | | 2 | | 226.25 | 0.82 | 175.1 | 21.38 | 500 | no | 0.65 | 0 | 0 | 0.32 |
| | D-129 | 1 | | 225.53 | 15.27 | 175.23 | 21.46 | 500 | no | 3.11 | 0.2 | 0.01 | 0.82 |
| | | 2 | | 224.23 | 0.82 | 100.72 | 12.41 | 500 | no | 0.95 | 0.1 | 0.01 | 0.32 |
| Example 6 | D-131 | 1 | Raw | 150.2 | 15.27 | 200.3 | 36.84 | 500 | no | 1.48 | 0.27 | 0.06 | 0.49 |
| | D-132 | 1 | palm oil # | 152.23 | 15.27 | 203.5 | 36.93 | 500 | no | 1.52 | 0.22 | 0.03 | 0.52 |
| | D-133 | 1 | | 149.89 | 15.27 | 201.3 | 37.10 | 500 | no | 1.68 | 0.25 | 0.05 | 0.48 |
| Example 7 | D-130 | 1 | ## | 121.1 | 151.52 | 201.1 | 13.28 | 1000 | no | 1.48 | 0.27 | 0.06 | 6.83 |

AV (Acid Value); mg KOH/g oil
(*) (**) 7% oleic acid added;
(***) Lab. Analysis: Water, 0.35%; AV 49.68 (Free Fatty Acids 24.88)
# Lab. Analysis: Water, 0.19%; AV 15.27 (Free Fatty Acids 7.63)
## Methanolic extract FFA from raw oil; AV = 151.52

TABLE 2
Flash evaporation

| | | | | Oil | | Flash evaporation | | | | | |
|---|---|---|---|---|---|---|---|---|---|---|---|
| Example | | | Raw | Amount | MeOH | MeOH | MeOH | Water | MG | DG | TG |
| no. | Charge | Step | material | (g) | (g) | (g) | (%) | (%) | (%) | (%) | (%) |
| Example 8 | B-139 | 1 | Raw | 221.3 | 175.2 | 148.1 | 84.6 | 0.95 | 4.99 | 0.24 | 0.03 |
| | | 2 | Palm oil | | | | | | 1.51 | 0.11 | 0 |
| | B-140 | 1 | | 225.2 | 175.5 | 146.6 | 83.5 | 1.1 | 4.68 | 0.29 | 0.05 |
| | B-141 | 1 | | 222.3 | 169.8 | 148.8 | 87.6 | 0.91 | | | |

AV (Acid Value); mg KOH/g oil;
# Lab. Analysis: Water, 0.19%; AV 15.27 (Free Fatty Acids 7.63)

### CONCLUSION

Generally we can conclude:
The esterification and transesterification is possible to perform in one step.
Raw material containing up to 25% of free fatty acid can be applied in described process, giving about 95% fatty acid methyl ester (FAME).
The yield of esterification is about 95%.

The fatty acid ester produced can be used for various purposes, particularly for a fuel, such as a diesel fuel, a fuel oil, or a base oil for a lubricant.

What is claimed is:

1. A process for producing a fatty acid ester comprising: reacting an oil or fat with an alcohol under subcritical conditions in a reactor without stirring and in the presence of a catalyst Ti(OR)₄.

2. The process according to claim 1, wherein the reaction is carried out under a pressure of 28-60 bars and a temperature of 200-240° C.

3. The process according to claim 1, wherein the reaction is carried out in only one or two stages.

4. The process according to claim 1 wherein, in, a first stage, oil and methanol are heated with 300-600 ppm of the catalyst.

5. The process according to claim 4, wherein 15-20 moles of methanol to 1 mol of oil is employed.

6. The process according to claim 5, wherein approximately 500 ppm of the catalyst is employed.

7. The process according to claim 1, further comprising: heating the oil or fat and alcohol for 25-35 minutes and at a temperature of 200-240° C.

8. The process according to claim 7, wherein the heating is performed for approximately 30 minutes at about 230° C.

9. The process according to claim 1, wherein the oil or fat is not pretreated.

10. The process according to claim 1, wherein a total reaction time is no greater than 60 minutes.

11. The process according to claim 1, wherein oil is reacted with the alcohol.

12. The process according to claim 11, wherein the oil has even 25% of free acids.

13. The process according to claim 1, wherein the alcohol is represented by the formula R—OH.

14. The process according to claim 13, wherein R is an alkyl group having 1 to 10 carbon atoms.

15. The process according to claim 1, wherein the oil or fat is a waste oil and fat.

16. The process according to claim 1, wherein the oil or fat is a waste edible oil.

17. The process according to claim 1, wherein R of the catalyst is selected from the group consisting of methyl, ethyl, isopropyl, n-buthyl, 2-ethylhexyl and octyleneglycole.
